(19) United States
(12) Patent Application Publication
(10) Pub. No.: US 2005/0209186 A1
(43) Pub. Date: Sep. 22, 2005

(54) Method for treating chronic myelogenous leukemia

(76) Inventor: John Lyons, Moraga, CA (US)

Correspondence Address: WILSON SONSINI GOODRICH & ROSATI, 650 PAGE MILL ROAD, PALO ALTO, CA, 94304-1050, US

(21) Appl. No.: 11/004,665

(22) Filed: Dec. 3, 2004

Related U.S. Application Data
(63) Continuation of application No. 10/071,849, filed on Feb. 7, 2002, now abandoned.

(51) Int. Cl. A61K 31/7072

(52) U.S. Cl. 514/49

(57) ABSTRACT

Methods, compositions and kits are provided for treating cancer associated with protein tyrosine kinase activity such as chronic myelogenous leukemia. In particular, a treatment method is provided comprising: administering to a patient having chronic myelogenous leukemia and a degree of resistance to imatinib mesylate, a therapeutically effective amount of a DNA methylation inhibitor which mitigates the imatinib mesylate resistance.

### CROSS-REFERENCE TO RELATED APPLICATION

This application is a continuation of U.S. application Ser. No. 10,071,849 filed on Feb. 7, 2002, which is incorporated herein by reference in its entirety and to which application we claim priority under 35 USC § 120.

### BACKGROUND OF THE INVENTION

1. Field of the Invention

The invention relates to methods, compositions, and kits for treating cancer associated with protein tyrosine kinase activity, and more particularly for treating chronic myelogenous leukemia.

2. Description of Related Art

Chronic Myelogenous Leukemia (CML) is a myeloproliferative disorder of a pluripotent hematopoietic stem cell with a particular cytogenetic abnormality, the Philadelphia chromosome. Faderl et al (1999) Ann. Intern. Med. 131:207-219. In childhood, it accounts for only 2 to 5% of all malignant disorders and presents as either of two distinct clinical entities, adult-type CML and juvenile CML. Adult-type CML of childhood is indistinguishable from that seen in older patients. However, juvenile CML is restricted to children and is Philadelphia chromosome negative. Grier and Civin (1998) in (Nathan and Oski, eds) Hematology of Infancy and Childhood, volume 2, 5th ed, W. B. Saunders Company, 34:1286-1459.

CML is a progressive, uniformly fatal disease in untreated patients. It is characterized by three distinct phases: a chronic phase lasting three to five years; an acute or accelerated phase lasting three to six months; and a brief blastic crisis phase. The progression of the disease to blast crisis results in rapid death due to infections, bleeding and leukemic organ infiltration.

Philadelphia chromosome, the characteristic cytogenetic abnormality of CML, results from a reciprocal chromosomal translocation, t(9;22)(q34;q11), in a hematopoietic stem cell. This translocation produces a fusion gene, termed Bcr-Abl, created by the juxtaposition of the abelson murine leukemia (Abl) protooncogene on chromosome 9 with a portion of the breakpoint cluster region (Bcr) gene on chromosome 22. The Bcr-Abl fusion protein's leukomogenic potential is derived from its constitutively activated tyrosine kinase activity, which causes a perturbation of stem cell function through unclear mechanisms. This activity results in interference with basic cellular processes, such as control of proliferation, adherence, and physiological death. More advanced stages of CML are also characterized by aberrant methylation of multiple genes, including the p15/Ink-4b cell-cycle regulator gene. Cortes et al (1997) Baillieres Clin Haematol 10(2):277-90. Aberrations in DNA methylation, whether general or site specific, are common in cancer and have important roles in tumor initiation, progression and resistance. Lubbert et al (2001) Br J Haematol 114(2):349-357.

Until recently, standard therapy for chronic phase CML consisted of conventional chemotherapy, interferon-alpha (with or without Ara-C), donor lymphocyte infusions and allogeneic bone marrow transplantation, each offering different risk-benefit trade-offs.

Overall, available data suggests the view that allogeneic bone marrow transplantation offers to eligible patients (children and young adults with an human leukocyte antigen-matched sibling donor) their best prospect for cure. However, bone marrow transplantation has certain limitations i.e., the availability of a suitable donor (10-40%), the risk of graft-vs.-host disease (8-60%) and a high rate of transplant-related mortality (20-40%).

Long-term follow-up of patients treated in large-scale randomized trials utilizing one or two of the above therapeutic modalities has shown a significant correlation between cytogenetic responses and prolonged survival. Silver et al (1999) Blood 94:1517-1536.

Imatinib mesylate is one of the recent therapeutic breakthroughs in the treatment of CML. Imatinib mesylate is a small molecule inhibitor of tyrosine kinase activity that results in a high response rate in CML. In the pivotal Phase II studies, nearly all patients (88%) with chronic phase CML achieved a complete hematologic response, and nearly half (49%) had a major cytogenetic response. Imatinib mesylate produced remissions in 63% of accelerated phase CML patients and 26% of blast phase patients. A complete cytogenetic response was seen in 30% of chronic phase CML, 14% of accelerated phase CML, and 5% of blast phase CML patients and was maintained for four weeks in 16%, 4%, and 1%, respectively. Novartis, Gleevec package insert T-2001-14 90012401.

Imatinib mesylate was approved by the FDA in May 2001 for the treatment of CML in all phases (after failure of interferon in the chronic phase). However, in blast phase CML, the responses to imatinib mesylate are usually of very short duration, and most patients manifest resistant/refractory disease within six months of therapy. Druker et al (2001) N. Engl. J. Med. 344: 1038-1042. Resistance to imatinib mesylate was associated with reactivation of Bcr-Abl and could be conferred by a single point substitution of threonine for isoleucine in the tyrosine kinase. Gorre et al (2001) Science 293: 2163. Consequently, there exists a need for compositions and methods for treating CML patients who are resistant to imatinib mesylate.

### SUMMARY OF THE INVENTION

A method is provided for treating a patient with imatinib mesylate in combination with a DNA methylation inhibitor. The method is preferably directed to a patient that has a degree of resistance to imatinib mesylate, the resistance being mitigated by the administration of the DNA methylation inhibitor. In particular, the method is directed to treating a disease state associated with activity of protein tyrosine kinase such as oncoprotein Bcr-Abl involved in chronic myelogenous leukemia (CML), platelet-derived growth factor (PDGF) receptor involved in prostate cancer and glioblastoma, and c-Kit involved in gastrointestinal stromal tumor (GIST) and small cell lung cancer (SCLC), as well as other types of cancer, where the combination treatment using imatinib mesylate and a DNA methylation inhibitor is synergistic.

In one embodiment, the method comprises: administering to the patient imatinib mesylate and a DNA methylation inhibitor.

In another embodiment, a method is provided for treating a patient having chronic myelogenous leukemia comprising: administering to a patient having chronic myelogenous leukemia and a degree of resistance to imatinib mesylate, a therapeutically effective amount of a DNA methylation inhibitor which mitigates the imatinib mesylate resistance.

In one variation, the patient has already manifested resistance to imatinib mesylate within 6 months of the treatment with imatinib mesylate as defined by no improvement in the prognosis or worsening of the prognosis.

In another embodiment, a method is provided for treating a patient having chronic myelogenous leukemia comprising: administering to a patient having chronic myelogenous leukemia and manifesting intolerance to imatinib mesylate, a therapeutically effective amount of a DNA methylation inhibitor which mitigates the imatinib mesylate intolerance.

In one variation, the patient has already manifested intolerance to imatinib mesylate within 6 months of the treatment with imatinib mesylate as defined by manifesting a symptom selected from the group consisting of hepatoxicity, fluid retention syndrome, neutropenia, hemorrhage, dyspepsia, dyspnea, diarrhea, muscle cramps, skin rash, fatigue, headache, nausea, vomiting, and thrombocytopenia.

In another embodiment, a method is provided for treating a patient having chronic myelogenous leukemia comprising: administering to the patient imatinib mesylate and a DNA methylation inhibitor.

In another embodiment, a method is provided for treating a patient having chronic myelogenous leukemia and a degree of resistance to imatinib mesylate comprising: administering to the patient imatinib mesylate and a DNA methylation inhibitor such that the patient's resistance to imatinib mesylate in the absence of the DNA methylation inhibitor is reduced.

In another embodiment, a method is provided for treating a patient having chronic myelogenous leukemia, comprising: administering to a patient in blast phase of chronic myelogenous leukemia a therapeutically effective amount of a DNA methylation inhibitor.

According to any of the above methods for treating chronic myelogenous leukemia, the patient's chronic myelogenous leukemia is optionally staged prior to administration: Staging the patient having chronic myelogenous leukemia optionally includes determining the number of blasts, promyelocytes, basophil, and platelets per liter of peripheral blood or bone marrow.

Also according to any of the above methods for treating chronic myelogenous leukemia, the DNA methylation inhibitor is optionally administered to the patient in the blast, chronic or accelerated phase of chronic myelogenous leukemia. In one variation, the method is performed when the patient in blast phase of chronic myelogenous leukemia has more than 30% blasts in peripheral blood or bone marrow.

Also according to any of the above methods for treating chronic myelogenous leukemia, the DNA methylation inhibitor may be administered by a variety of routes, including but not limited to orally, parenterally, intraperitoneally, intravenously, intraarterially, transdermally, sublingually, intramuscularly, rectally, transbuccally, intranasally, liposomally, via inhalation, vaginally, intraoccularly, via local delivery, subcutaneously, intraadiposally, intraarticularly, or intrathecally.

According to any of the above methods, it is noted that administering imatinib mesylate and the DNA methylation inhibitor to the patient may comprise administering imatinib mesylate to the patient for a period of time prior to the administration of the DNA methylation inhibitor, administering the DNA methylation inhibitor to the patient for a period of time prior to the administration of imatinib mesylate, or initiating administration of the DNA methylation inhibitor and imatinib mesylate to the patient at the same time. It is noted that the method may also comprise administering imatinib mesylate and the DNA methylation inhibitor to the patient at the same time for at least a portion of the time that the drugs are administered.

According to any of the above methods, in one variation, imatinib mesylate is administered to the patient at a dose of 100-800 mg/day, optionally at a dose of 200-400 mg/day, and optionally at a dose of 500-800 mg/day. Such administrations may optionally last for a period of at least 2, 4, 6, 8, 10 or more days.

Also according to any of the above methods, in one variation, the DNA methylation inhibitor is administered to the patient via an intravenous infusion per day at a dose ranging from 1 to 100 mg/m2, optionally at a dose ranging from 2 to 50 mg/m2, and optionally at a dose ranging from 5 to 20 mg/m2.

Also according to any of the above methods, the DNA methylation inhibitor may optionally be a cytidine analog such as cytosine arabinoside. In one variation, the cytidine analog is decitabine.

In one particular variation, the DNA methylation inhibitor is decitabine and is administered intravenously or subcutaneously. In a further particular variation, decitabine is administered to the patient via an intravenous infusion per day at a dose ranging from 1 to 100 mg/m2, optionally ranging from 2 to 50 mg/m2 and optionally ranging from 5 to 20 mg/m2.

In one example, decitabine is administered to the patient via an intravenous infusion per day for at least 3 days per treatment cycle at a dose ranging from 1 to 100 mg/m2. In a further example, decitabine is administered to the patient via an intravenous infusion at a dose ranging from 5 to 20 mg/m2 for 1 hour per day for 5 consecutive days for 2 weeks per treatment cycle.

Compositions are also provided. In one embodiment, a composition is provided that comprises a DNA methylation inhibitor and imatinib mesylate. The DNA methylation inhibitor may optionally be a cytidine analog such as cytosine arabinoside. In one variation, the cytidine analog is decitabine. In another variation, the composition is formulated for intravenous, inhalation, oral, or subcutaneous administration.

### DETAILED DESCRIPTION OF THE INVENTION

In general, the present invention provides novel therapeutics and methods for treating diseases associated with abnormal cell proliferation caused by activation of oncogenes and/or suppression of tumor suppressors. In particular, methods are provided for treating a disease state associated with activity of protein tyrosine kinase such as oncoprotein Bcr-Abl involved in chronic myelogenous leukemia (CML), platelet-derived growth factor (PDGF) receptor involved in prostate cancer and glioblastoma, and c-Kit involved in gastrointestinal stromal tumor (GIST) and small cell lung cancer (SCLC), as well as other types of cancer.

In one aspect of the present invention, a monotherapy with a DNA methylation inhibitor is provided for treating a CML patient who has a degree of resistance to imatinib mesylate, especially in accelerated or blast phase of CML. This monotherapy may also be used to treat patients having other diseases but manifesting resistance to the treatment of imatinib mesylate.

In another aspect of the present invention, a combination therapy is provided for treating various types of cancer associated with protein tyrosine kinase activity, such as CML, prostate cancer, glioblastoma, GIST and SCLC.

The inventors believes that a DNA methylation inhibitor such as decitabine should reactivate certain genes which participate in the pathways of protein tyrosine kinase but with their functions effected by methylation, presumably in the promoter regions. Loss of these gene functions could lead to elevated expression and/or activity of protein tyrosine kinase, resulting worse prognosis of the disease. Since imatinib mesylate has a strong inhibitory effect on protein tyrosine kinase, a treatment combining the use of imatinib mesylate and decitabine would have a synergistic effect via targeting different genes in the signal transduction pathways. Further, lower doses of these two drugs may be used in the combination therapy to reduce side effects associated with the monotherapy with either one of these two drugs.

1. Imatinib Mesylate

Imatanib mesylate is a protein tyrosine kinase inhibitor that inhibits the Bcr-Abl tyrosine kinase created by the Philadelphia chromosome abnormality in CML. Imatanib mesylate achieves this inibitory result through binding to the adenosine triphosphate-binding site of the Bcr-Abl tyrosine kinase, which prevents phosphorylation of substrates and related malignant transformation. Through inhibition of this kinase, it is believed that imatib mesylate inhibits cell proliferation and induces apoptosis. T. Schindler et al (2000) Science 289:1938-1942.

Imatinib mesylate is indicated for treatment of CML patients in blast phase, accelerated phase, or in chronic phase after failure of interferon-alpha therapy. Present dosages recommended for treatment with imatinib mesylate are 400 mg/day for patients with chronic phase CML and 600 mg/day for patients with accelerated phase or blast phase CML. In the event of disease progression, failure to achieve a satisfactory hematologic response after at least 3 months of treatment; or loss of a previously achiever hematologic response, the dose of imatinib mesylate may be increased. Treatment dosage may be increased in patients with chronic phase CML from 400 mg/day to 600 mg/day in the absence of severe adverse drug reaction and sever non-leukemia related neutropenia or thrombocytopenia. Simarlarly, treatment dosage may be increased in patients with chronic phase CML from 600 mg/day to 800 mg/day. Novartis, Gleevec package insert T-2001-14 90012401.

However, many CML patients do not respond or lose response to treatment with imatinib mesylate. This is particularly the case in blast phase CML, the responses to imatinib mesylate are usually of very short duration, and most patients manifest resistant/refractory disease within six months of therapy. Druker et al (2001) N. Engl. J. Med. 344: 1038-1042. According to the present invention, it is believed that DNA methylation inhibitors can be used to treat CML patients that are resistant to the treatment with imatinib mesylate.

2. DNA Methylation Inhibitors

Decitabine, 5-aza-2′-deoxycytidine, is an antagonist of its related natural nucleoside, deoxycytidine. The only structural difference between these two compounds is the presence of a nitrogen at position 5 of the cytosine ring in decitabine as compared to a carbon at this position for deoxycytidine. Two isomeric forms of decitabine can be distinguished. The -anomer is the active form. The modes of decomposition of decitabine in aqueous solution are (a) conversion of the active -anomer to the inactive -anomer (Pompon et al. (1987) J. Chromat. 388:113-122); (b) ring cleavage of the aza-pyrimidine ring to form N-(formylamidino)-N′-D-2′-deoxy-(ribofuranosy)-urea (Mojaverian and Repta (1984) J. Pharm. Pharmacol. 36:728-733); and (c) subsequent forming of guanidine compounds (Kissinger and Stemm (1986) J. Chromat. 353:309-318).

Decitabine possesses multiple pharmacological characteristics. At a molecular level, it is capable of specifically inhibiting cell growth at S phase and DNA methylation. At a cellular level, decitabine can induce cell differentiation and exert hematological toxicity. Despite having a short half life in vivo, decitabine has excellent tissue distribution.

The most prominent function of decitabine is its ability to specifically and potently inhibit DNA methylation. For example, in the methylation of cytosine in CpG islands, methylation of cytosine to 5-methylcytosine occurs at the level of DNA. Inside the cell, decitabine is first converted into its active form, the phosphorylated 5-aza-deoxycytidine, by deoxycytidine kinase which is primarily synthesized during the S phase of the cell cycle. The affinity of decitabine for the catalytical site of deoxycytidine kinase is similar to the natural substrate, deoxycytidine. Momparler et al. (1985) 30:287-299. After conversion to its triphosphate form by deoxycytidine kinase, decitabine is incorporated into replicating DNA at a rate similar to that of the natural substrate, dCTP. Bouchard and Momparler (1983) Mol. Pharmacol. 24:109-114.

Incorporation of decitabine into the DNA strand has a hypomethylation effect. Each class of differentiated cells has its own distinct methylation pattern. After chromosomal duplication, in order to conserve this pattern of methylation, the 5-methylcytosine on the parental strand serves to direct methylation on the complementary daughter DNA strand. Substituting the carbon at the 5 position of the cytosine for a nitrogen interferes with this normal process of DNA methylation. The replacement of 5-methylcytosine with decitabine at a specific site of methylation produces an irreversible inactivation of DNA methyltransferase, presumably due to formation of a covalent bond between the enzyme and decitabine. Juttermann et al. (1994) Proc. Natl. Acad. Sci. USA 91:11797-11801. By specifically inhibiting DNA methyltransferase, the enzyme required for methylation, the aberrant methylation of the tumor suppressor genes can be prevented.

3. Treatment of Different Disease States

Described herein are several different disease states which may be treated by the combination therapy methods and compositioned provided herein. It is noted that other disease states that may be treated with imatinib mesylate may also be treated with the combination of imatinib mesylate and a DNA methylation inhibitor where the DNA methylation inhibitor synergistically renders the imatinib mesylate more effective, for example by reducing resistance to imatinib mesylate that the patient may naturally have or may develop over time.

### A. Chronic Myelogenous Leukemia

Unlike other forms of leukemia, CML is a homogeneous disease. Almost all patients with CML (90%) have the same chromosomal abnormality in their leukemic cells. In up to 40% of CML patients, the disease progresses directly from the chronic to the blastic phase. Cortes and Kantarjian (1998) in Cancer management: a multidisciplinary approach, 2d ed. Huntington, N.Y.: Publisher Research Management, Inc, p306-15. Blast transformation typically occurs at 3 to 5 years, but onset is random and may be observed at the time of initial diagnosis. Common features for chronic phase CML patients include fatigue, weight loss, and signs or symptoms of splenomegaly (e.g., left upper quadrant pain, abdominal fullness, a palpable mass), which occurs in up to 70% of patients. Cortes et al (1996) Am J Med 100(5):555-70. Patients with extreme elevations in white blood cell count (hyperleukocytosis) may demonstrate signs or symptoms of leukostasis, including mental status changes, focal neurologic deficits, tinnitus, dyspnea, and priapism. Rtinal hemorrhages or other bleeding manifestations may appear irrespective of platelet counts. This is attributable to the qualitative platelet dysfunction that is common in CML. Goldman (1997) BMJ (Clin Res Ed) 314:657-60.

As CML progresses from chronic phase to accelerated phase, the patient may experience fever, night sweats, weight loss, and progressive splenomegaly. More often, however, there is no significant change in symptoms, and onset is heralded by hematologic progression (e.g., worsening of blood counts) or cytogenetic evolution (e.g., development of new chromosomal abnormalities). As CML progresses from the accelerated phase to blast phase, the patient may experience fever, weight loss, night sweats, bone pain, and constitutional symptoms. Lymphadenopathy, leukemia cutis, central nervous system disease, and bleeding secondary to progressive thrombocytopenia also may occur. Hughes and Goldman (1995) in Hematology: basic principles and practice, 2d ed. New York: Churchill Livingstone, p1142-59.

CML staging involves the use of a system of categorization to describe the seriousness of the CML and attempts to put patients with similar prognosis and treatment in the same staging group. Staging is important in developing CML treatment strategies as it allows doctors to compare the efficacy of different treatments for patients with similar conditions, and aids in the determination of treatment decisions. Staging systems for CML are generally based on clinical features with demonstrated prognostic significance, including age, size of spleen, percentage of circulating and marrow blasts, degree of basophilia, extent of thrombocytosis, and presence of atypical chromosomal abnormalities. Faderl, S., Talpaz, M., Estrov, Z., and Kantarjian, H. M. Chronic myelogenous leukemia: biology and therapy. Ann. Intern. Med., 131: 207-219, 1999; Grier H E, Civin C I. Myeloid Leukemias, Myelodysplasia, and Myeloproliferative Diseases in Children. In (Nathan and Oski, eds) Hematology of Infancy and Childhood, volume 2”. 5th Edition, W. B. Saunders Company, 1998; 34:1286-1459; Santini, V., Kantarjian, H. M., and Issa, J. P. Changes in DNA methylation in neoplasia: pathophysiology and therapeutic implications. Ann. Intern. Med., 134: 573-586, 2001; Lubbert M, Wijermans P, Kunzman R, et al. Cytogenetic responses in high-risk myelodysplastic syndrome following low-dose treatment with the DNA methylation inhibitor 5-aza-2′-deoxycytidine. Br J Haematol 2001 August; 114(2):349-357. In addition, specific response to interferon alpha-2b therapy is considered to be a particularly sensitive predictor of long-term survival following treatment. Goldman (1997) Baillieres Clin Haematol 10(2):405-21.

Prior to the administration of a DNA methylation inhibitor, CML patients may optionally be staged to determine the severity of the CML. Staging may include determining the number of blasts, promyelocytes, basophil, and platelets per liter of peripheral blood or bone marrow.

Patients with CML in the chronic phase have all of the following conditions: less than 15% blasts in the peripheral blood or bone marrow; less than 30% blasts and promyleocytes in the peripheral blood or bone marrow; less than 20% basophils in the peripheral blood; 100 times 10 supra 9 per liter platelets; and no extramedullary involvement other than liver or spleen. Patients with CML in the accelerated phase have at least one of the following conditions: 15% to less than 30% blasts in the peripheral blood or bone marrow; 30% blasts and promyleocytes in the peripheral blood or bone marrow (but less than 30% blasts in the peripheral blood or bone marrow); 20% basophils in the peripheral blood; or less than 100 times 10 supra 9 per liter platelets.

Blast phase CML patients have at least 30% blasts and promyleocytes in the peripheral blood or bone marrow, or extramedullary involvement other than liver or spleen.

Preferable blast phase CML patients treated using the compositions and methods of the present invention are two years of age or older, have histologically confirmed diagnosis of blast phase CML, were previously treated with imatinib mesylate with no response or resulted in loss of response, and have bilirubin levels greater than or equal to 1.5 times the upper limit of normal, serum glutamic-oxaloacetic transaminase and serum glutamic-pyruvic transaminase levels greater than or equal to 2.5 times the upper limit of normal, and serum creatine levels greater than or equal to 1.5 times the upper limit of normal.

Patients with CML in chronic, accelerated or blast phase, who are resistant to standard therapy generally have a relatively poor prognosis with a rapid progression to blast crisis, bone marrow failure and death. To measure the success of a therapeutic treatment of CML, hematologic responses are determined. A complete hematologic response is defined as maintaining the following conditions for four weeks: less than or equal to 5% blasts in bone marrow; no peripheral blood blasts; absolute neutrophil count of greater than 1.5 times 10 supra 9 per liter; platelet count of greater than 100 times 10 supra 9 per liter; and no extramedullary involvement.

The Philadelphia chromosome, with the Bcr-Abl oncogene detectable at the molecular level, is present at diagnosis in 95% of patients. Optionally, complete hematologic responses are further classified according to suppression of the Philadelphia chromosome (Ph). For example, a patient with complete hematologic response and greater than 65% Ph positive is classified as no cytogenetic response. Patient responses that are 36% to 65% Ph positive are classified as minimal cytogenetic responses, 1% to 35% Ph positive are partial cytogenetic responses, and 0% Ph positive are complete cytogenetic responses.

A partial hematologic response is defined as maintaining the following conditions for four weeks: less than or equal to 5% blasts in bone marrow; no peripheral blood blasts; absolute neutrophil count of less than 1.5 times 10 supra 9 per liter; and platelet count of less than 100 times 10 supra 9 per liter.

A hematologic improvement is defined as maintaining the following conditions for four weeks: less 15% blasts in bone marrow and peripheral blood; less than 40blasts and promyeloctes in peripheral blood and blood marrow; less than 20% basophils in peripheral blood; and no extramedullary involvement other than liver or spleen.

CML disease progression is associated with hypermethylaton of a promoter region within Bcr-Abl. J-P Issa et al (1999) Blood 93:2075-2080. In mammalian cells, approximately 3% to 5% of the cytosine residues in genomic DNA are present as 5-methylcytosine. Ehrlich et al (1982) Nucleic Acid Res. 10:2709-2721. This modification of cytosine takes place after DNA replication and is catalyzed by DNA methyltransferase using S-adenosyl-methionine as the methyl donor. Approximately 70% to 80% of 5-methylcytosine residues are found in the CpG sequence. Bird (1986) Nature 321:209-213. This sequence, when found at a high frequency, in the genome, is referred to as CpG islands. Unmethylated CpG islands are associated with housekeeping genes, while the islands of many tissue-specific genes are methylated, except in the tissue where they are expressed. Yevin and Razin (1993) in DNA Methylation: Molecular Biology and Biological Significance. Basel: Birkhauser Verlag, p523-568. This methylation of DNA has been proposed to play an important role in the control of expression of different genes in eukaryotic cells during embryonic development. Consistent with this hypothesis, inhibition of DNA methylation has been found to induce differentiation in mammalian cells. Jones and Taylor (1980) Cell 20:85-93.

Methylation of DNA in the regulatory region of a gene can inhibit transcription of the gene. This may be because 5-methylcytosine protrudes into the major groove of the DNA helix, which interferes with the binding of transcription factors.

The methylated cytosine in DNA, 5-methylcytosine, can undergo spontaneous deamination to form thymine at a rate much higher than the deamination of cytosine to uracil. Shen et al. (1994) Nucleic Acid Res. 22:972-976. If the deamination of 5-methylcytosine is unrepaired, it will result in a C to T transition mutation. For example, many “hot spots” of DNA damages in the human p53 gene are associated with CpG to TpG transition mutations. Denissenko et al. (1997) Proc. Natl. Acad. Sci. USA 94:3893-1898.

### B. Treatment of Chronic Myelogenous Leukemia

In a particular embodiment, a method is provide for treating CML. It has been found that unchecked production of tyrosine kinase Bcr-Abl leads to excessive levels of white blood cells in the blood and bone marrow, but disrupts the normal production of white blood cells; and imatinib mesylate works specifically to block the activity of Bcr-Abl tyrosine kinase. However, methylation of Bcr-Abl has been found to correlate with progression with CML: hypermethylation in 24-68% of CML patients in the chronic phase, while patients in the accelerated phase and blast crisis had hypermethylation frequencies at 73% and 80%, respectively. Issa et al. (1999) Blood 93:2075-2080.

Other than Bcr-Abl gene, multiple genes, including the p15/Ink-4b cell-cycle regulator gene, are found to contain aberrant methylation in more advanced stages of CML. Cortes et al (1997) Baillieres Clin Haematol 10(2):277-90. Aberrations in DNA methylation, whether general or site specific, are common in cancer and have important roles in tumor initiation, progression and resistance. Lubbert et al (2001) Br J Haematol 114(2):349-357. Methylation of the p15 promoters is associated with progression of CML. Nguyen et al. (2000) Blood 95:2990-2992.

The inventor believes that hypermethylation of p15 would lead to loss of its tumor suppression function and accelerates transformation of Bcr-Abl, ultimately leading to resistance to the treatment of CML with imatinib mesylate. Administering a DNA methylation inhibit to a CML patient may re-establish the tumor suppression functions of p15, which, in turn, leads to sensitization of the patient to the treatment with imatinib mesylate.

The present invention provides a method of treating a patient having chronic myelogenous leukemia, comprising: administering to a patient having chronic myelogenous leukemia but resistant to the treatment with imatinib mesylate a therapeutically effective amount of a DNA methylation inhibitor. In one variation, the patient has already manifested resistance to imatinib mesylate within 6 months of the treatment with imatinib mesylate as defined by no improvement in the prognosis or worsening of the prognosis.

The DNA methylation inhibitor may be optionally administered to the patient in the blast, chronic or accelerated phase of chronic myelogenous leukemia. In one embodiment of the present invention, a method is provided for treating patients having blast phase CML, where prior to treatment, the patient's CML is staged. The method comprises administering to a patient suffering from blast phase CML, after the CML has been staged, a DNA methylation inhibitor. Optinally, staging the CML includes determining the number of blasts, promyelocytes, basophil, and platelets per liter of peripheral blood or bone marrow. Optionally, the patient in blast phase of CML has more than 30% blasts in the peripheral blood or bone marrow.

A method is also provided for treating a patient having chronic myelogenous leukemia but manifesting intolerance to imatinib mesylate. The method comprises: administering to the patient a therapeutically effective amount of a DNA methylation inhibitor which mitigates the imatinib mesylate intolerance.

Preferably the DNA methylation inhibitor is administered to a patient who has already manifested intolerance to imatinib mesylate within 6 months of the treatment with imatinib mesylate. The patient's intolerance to imatinib mesylate can be defined by manifesting symptoms or adverse effects such hepatoxicity, fluid retention syndrome, neutropenia, hemorrhage, dyspepsia, dyspnea, diarrhea, muscle cramps, skin rash, fatigue, headache, nausea, vomiting, and thrombocytopenia.

A method is also provided for treating a patient having chronic myelogenous leukemia, comprising: co-administering to the patient imatinib mesylate and decitabine such that the patient's resistance to imatinib mesylate in the absence of decitabine is reduced.

In one variation, imatinib mesylate is administered to the patient at a dose of 100-800 mg/day. For chronic phase CML patients, imatinib mesylate is preferentially administered at a dose of 200-400 mg/day. For accelerated or blast phase CML patients, imatinib mesylate is preferably administered at a dose of 500-800 mg/day.

In another embodiment of the present invention, a composition is provided that comprises a DNA methylation inhibitor and imatinib mesylate. The DNA methylation inhibitor may optionally be a cytidine analog such as cytosine arabinoside. In one variation, the cytidine analog is decitabine. In one variation, the composition is formulated for intravenous or subcutaneous administration. The inventive combination of DNA methylation inhibitor and imatinib mesylate may be administered by a variety of routes, and may be administered or coadministered in any conventional dosage form. Coadministration in the context of this invention is defined to mean the administration of more than one therapeutic in the course of a coordinated treatment to achieve an improved clinical outcome. Such coadministration may also be coextensive, that is, occurring during overlapping periods of time. For example, the DNA methylation inhibitor may be administered to a patient before, concomitantly, or after imatinib mesylate is administered. In one variation, the patient is treated first with imatinib mesylate and then treated with the DNA methylation inhibitor (e.g., decitabine).

### C. Other Disease States

Compositions and methods of the present invention may also be used for treating a patient with diseases other than CML, especially diseases associated with activity of protein kinase, and more particularly protein tyrosine kinase.

For example, the compositions and methods of the present invention may be used to treat diseases associated with tyrosine kinase activity of platelet-derived growth factor (PDGF) receptor such as prostate cancer and glioblastoma. PDGF, such as PDGF A and B, signals through a cell surface tyrosine kinase receptor (PDGF-R) to stimulate various cellular functions including growth, proliferation, and differentiation. George (2001) Semin. Oncol. 28 (5 Suppl 17):27-33. PDGF expression has been shown in various different types of solid tumors, such as prostate cancer and glioblastoma. Both PDGF-R and Bcr-Abl signal through the Ras/MAP kinase pathway.

The inventor believes that while imatinib mesylate can also inhibit tyrosine kinase activity of PDGF-R, as in CML loss of p15 tumor suppression function may lead to transformation of PDGF-R, ultimately leading to resistance to imatinib mesylate treatment. By using the composition and methods of the present invention, diseases associated with tyrosine kinase activity of PDGF-R can be treated more efficaciously and with significantly reduced adverse effects.

The compositions and methods of the present invention may also be used to treat diseases associated with tyrosine kinase activity of the transmembrane protein c-Kit such as gastrointestinal stromal tumor (GIST) and small cell lung cancer (SCLC). c-Kit is defined by the CD117 antigen and is the product of the c-kit proto-oncogene. Activating or gain-of-function mutations in the c-kit gene have been identified in the majority of GISTs. As a result, Kit is constitutively expressed to provide growth and survival signals to GIST cells, which are crucial to the pathogenesis of the disease. So far, GIST has been found to be the most common mesenchymal tumor of the GI tract and resistant to chemotherapy and radiation treatment. Recently, the US Food and Drug Administration (FDA) has approved imatinib mesylate (or GLEEVAC®) for the treatment of patient with KIT (or CD117)-positive unresectable and/or metastatic magligant GISTs.

The inventor believes that while imatinib mesylate can suppress GIST growth by inhibiting the tyrosine kinase activity of c-Kit, mutations in c-kit may render the disease less responsive to the treatment with imatinib mesylate. In addition, hypermethylation in the genes participating in the signal transduction pathway of c-Kit would also lead to transformation of c-Kit, ultimately leading to resistance to imatinib mesylate treatment. By targeting c-Kit and DNA hypermethylation using the combination therapy of the present invention, diseases associated with tyrosine kinase activity of c-Kit can be treated more efficaciously and with significantly reduced adverse effects.

4. Routes of Delivery, Formulations and Kits

The DNA methylation inhibitor employed in the present invention may be administered or coadministered in any conventional dosage form. For example, the inhibitor be administered or coadministered parenterally, orally, intraperitoneally, intravenously, intraarterially, transdermally, sublingually, intramuscularly, rectally, transbuccally, intranasally, liposomally, via inhalation, vaginally, intraoccularly, via local delivery by catheter or stent, subcutaneously, intraadiposally, intraarticularly, or intrathecally.

In a preferred embodiment, decitabine is administrated to a patient by injection, including intravenous or subcutaneous injection, such as bolus intravenous injection, continuous intravenous infusion and intravenous infusion over 1 hour. For example, decitabine may administered into the patient via an 1-24 hour intravenous infusion per day for 3-5 days per treatment cycle at a dose preferably ranging from 1-100 mg/m2, more preferably ranging from 2-50 mg/m2, and most preferably from 5-20 mg/m2. The preferred dosage below 50 mg/m2 for decitabine is considered to be much lower than that used in conventional chemotherapy for cancer.

In another embodiment, decitabine is administered via intravenous infusion at a dose ranging from 1 to 100 mg/m2 per day for at least 3 days per treatment cycle. In yet another embodiment, decitabine is administered via intravenous infusion at a dose ranging from 5 to 20 mg/m2 for 1 hour per day for 5 consecutive days for 2 weeks per treatment cycle.

The DNA methylation inhibitors employed in the invention may also be administered or coadministered in slow release dosage forms. Furthermore, the DNA methylation inhibitors may be administered or coadministered with conventional pharmaceutical excipients and additives.

Decitabine may be supplied as sterile powder for injection, together with buffering salt such as potassium dihydrogen and pH modifier such as sodium hydroxide. This formulation is preferably stored at 2-8° C., which should keep the drug stable for at least 2 years. This powder formulation may be reconstituted with 10 ml of sterile water for injection. This solution may be further diluted with infusion fluid known in the art, such as 0.9% sodium chloride injection, 5% dextrose injection and lactated ringer's injection. It is preferred that the reconstituted and diluted solutions be used within 4-6 hours for delivery of maximum potency.

The inventive combination of DNA methylation inhibitor and imatinib mesylate may be used in the form of kits. The arrangement and construction of such kits is conventionally known to one of skill in the art. Such kits may include containers for containing the inventive combination of therapeutic agents and/or compositions, and/or other apparatus for administering the inventive combination of therapeutic agents and/or compositions.

### EXAMPLES

### Example 1

Patients with CML receive decitabine at 15 mg/m2 IV over one hour on five consecutive days each week (e.g., Monday through Friday) for two weeks, followed by four weeks rest. For patients who achieve and remain in remission, treatment may continue so long as no adverse drug experiences occur and there is no evidence of disease progression or relapse.

Since the effect of decitabine may be delayed for up to two cycles, patients with high white blood cell counts (>10×109 per liter) at study entry may optionally receive hydroxyurea (HU) 1-3 g orally daily concomitantly with the first two cycles of decitabine only. HU may be continued until the end of dosing in the second cycle of decitabine (e.g., the 13th day of the second cycle) or until the white blood cell count falls below 10×109 per liter during the second cycle. This is done to minimize the risks of complications due to hyperleukocytosis while awaiting a response. In general, HU can be given a 1 g daily if the white blood cell count is between 10-40×109 per liter, 2 g daily if the white blood cell count is 40-50×109 per liter, and 3 g daily if the white blood cell count is above 50×109 per liter. Other dose schedules may be used as indicated. Children with white blood cell counts above 10-20×109 per liter can be treated with HU at 10-20 mg/kg/day. The use of anagrelide is allowed for platelets >600×109 per liter, but is generally discontinued prior to decitabine dosing in the third cycle.

Patients achieving an unconfirmed complete or partial hematological response, or a hematologic improvement, will continue to receive therapy every six weeks until they show evidence of disease progression or relapse.

The second and subsequent cycles of decitabine will be instituted when patients have recovered from hematologic or non-hematological toxicity. Hematologic recovery is generally an absolute neutrophil count >1×109 per liter or to baseline and platelets to >150×109 per liter, untransfused for the preceding week.

The decitabine dose may be escalated incrementally by 25% to reduce the absolute neutrophil count to between 1×109 per liter and 1.5×109 per liter and a platelet count to between 50×109 per liter and 100×109 per liter with each cycle. The decitabine dose may be decreased incrementally by 25% in the face of grade 4 hematologic toxicity or grade 3 or 4 non-hematological toxicity, other than alopecia.

It will be apparent to those skilled in the art that various modifications and variations can be made in the present invention without departing from the spirit or scope of the invention. Thus, it is intended that the present invention cover the modifications and variations of this invention provided they come within the scope of the appended claims and their equivalents. Additionally, the above examples are provided for the purpose of illustrating the claimed invention, and should not be construed so as to limit the scope of the claimed invention.

1. A treatment method comprising:
administering to a patient having chronic myelogenous leukemia and a degree of resistance to imatinib mesylate, a therapeutically effective amount of a DNA methylation inhibitor which mitigates the imatinib mesylate resistance.

2. The method of claim 1, wherein the DNA methylation inhibitor is a cytidine analog.

3. The method of claim 2, wherein the cytidine analog is cytosine arabinoside.

4. The method of claim 2, wherein the cytidine analog is decitabine.

5. The method of claim 1, wherein is administered by a route selected from the group consisting of the DNA methylation inhibitor orally, parenterally, intraperitoneally, intravenously, intraarterially, transdermally, sublingually, intramuscularly, rectally, transbuccally, intranasally, liposomally, via inhalation, vaginally, intraoccularly, via local delivery, subcutaneously, intraadiposally, intraarticularly, and intrathecally.

6. The method of claim 1, wherein the DNA methylation inhibitor is decitabine and is administered intravenously or subcutaneously.

7. The method of claim 6, wherein decitabine is administered to the patient via an intravenous infusion per day at a dose ranging from 1 to 100 mg/m2.

8. The method of claim 6, wherein decitabine is administered to the patient via an intravenous infusion per day at a dose ranging from 2 to 50 mg/m2.

9. The method of claim 6, wherein decitabine is administered to the patient via an intravenous infusion per day at a dose ranging from 5 to 20 mg/m2.

10. The method of claim 6, wherein decitabine is administered to the patient via an intravenous infusion per day for at least 3 days per treatment cycle at a dose ranging from 1 to 100 mg/m2.

11. The method of claim 6, wherein decitabine is administered to the patient via an intravenous infusion at a dose ranging from 5 to 20 mg/m2 for 1 hour per day for 5 consecutive days for 2 weeks per treatment cycle.

12. The method of claim 1, wherein the DNA methylation inhibitor is administered to the patient in blast phase of chronic myelogenous leukemia.

13. The method of claim 1, wherein the DNA methylation inhibitor is administered to the patient in chronic phase of chronic myelogenous leukemia.

14. The method of claim 1, wherein the DNA methylation inhibitor is administered to the patient in accelerated phase of chronic myelogenous leukemia.

15. The method of claim 1, wherein the patient has manifested resistance to imatinib mesylate within 6 months of the treatment with imatinib mesylate as defined by no improvement in the prognosis or worsening of the prognosis.

16. In a treatment method for a patient having chronic myelogenous leukemia and a degree of resistance to imatinib mesylate, the improvement comprising administering a therapeutically effective amount of a DNA methylation inhibitor which mitigates the imatinib mesylate resistance.

17. The method of claim 16, wherein the DNA methylation inhibitor is a cytidine analog.

18. The method of claim 17, wherein the cytidine analog is cytosine arabinoside.

19. The method of claim 17, wherein the cytidine analog is decitabine.

20. The method of claim 16, wherein imatinib mesylate is administered to the patient for a period of time prior to the administration of the DNA methylation inhibitor.

21. The method of claim 16, wherein imatinib mesylate is administered to the patient for a period of time after the administration of the DNA methylation inhibitor.

22. The method of claim 16, wherein the DNA methylation inhibitor is administered to the patient for a period of time prior to the administration of the imatinib mesylate.

23. The method of claim 16, wherein the DNA methylation inhibitor is administered to the patient for a period of time after the administration of the imatinib mesylate.

24. The method of claim 16, wherein the DNA methylation inhibitor is administered at the same time for at least a portion of the time that the imatinib mesylate is administered.

25. The method of claim 16, wherein is administered by a route selected from the group consisting of the DNA methylation inhibitor orally, parenterally, intraperitoneally, intravenously, intraarterially, transdermally, sublingually, intramuscularly, rectally, transbuccally, intranasally, liposomally, via inhalation, vaginally, intraoccularly, via local delivery, subcutaneously, intraadiposally, intraarticularly, and intrathecally.

26. The method of claim 16, wherein the DNA methylation inhibitor is decitabine and is administered intravenously or subcutaneously.

27. The method of claim 26, wherein decitabine is administered to the patient via an intravenous infusion per day at a dose ranging from 1to 100 mg/m2.

28. The method of claim 26, wherein decitabine is administered to the patient via an intravenous infusion per day at a dose ranging from 2 to 50 mg/m2.

29. The method of claim 26, wherein decitabine is administered to the patient via an intravenous infusion per day at a dose ranging from 5 to 20 mg/m2.

30. The method of claim 26, wherein decitabine is administered to the patient via an intravenous infusion per day for at least 3 days per treatment cycle at a dose ranging from 1 to 100 mg/m2.

31. A treatment method for treating a patient having chronic myelogenous leukemia, comprising:
administering to the patient imatinib mesylate and decitabine such that the patient's resistance to imatinib mesylate in the absence of decitabine is reduced.

32. The method of claim 31, wherein imatinib mesylate is administered to the patient for a period of time prior to the administration of the decitabine.

33. The method of claim 31, wherein imatinib mesylate is administered to the patient for a period of time after the administration of the decitabine.

34. The method of claim 31, wherein decitabine is administered to the patient for a period of time prior to the administration of the imatinib mesylate.

35. The method of claim 31, wherein decitabine is administered to the patient for a period of time after the administration of the imatinib mesylate.

36. The method of claim 31, wherein decitabine is administered at the same time for at least a portion of the time that the imatinib mesylate is administered.

37. The method of claim 31, wherein imatinib mesylate is administered to the patient at a dose of 100-800 mg/day.

38. The method of claim 31, wherein the patient is in chronic phase of chronic myelogenous leukemia and imatinib mesylate is administered to the patient at a dose of 200-400 mg/day.

39. The method of claim 31, wherein the patient is in blast or accelerated phase of chronic myelogenous leukemia and imatinib mesylate is administered to the patient at a dose of 500-800 mg/day.

40. The method of claim 31, wherein decitabine is administered to the patient via an intravenous infusion per day at a dose ranging from 1 to 100 mg/m2.

41. The method of claim 31, wherein decitabine is administered to the patient via an intravenous infusion per day at a dose ranging from 2 to 50 mg/m2.

42. The method of claim 31, wherein decitabine is administered to the patient via an intravenous infusion per day at a dose ranging from 5 to 20 mg/m2.

43. A method for treating a patient having chronic myelogenous leukemia, comprising:
administering to a patient in blast phase of chronic myelogenous leukemia a therapeutically effective amount of a DNA methylation inhibitor in combination with imatinib mesylate.

44. The method of claim 43, where prior to administering, the patient's chronic myelogenous leukemia is staged.

45. The method of claim 44, wherein staging the patient having chronic myelogenous leukemia includes determining a number of blasts, promyelocytes, basophil, and platelets per liter of peripheral blood or bone marrow.

46. The method of claim 43, wherein administration is performed when the patient is in blast phase of chronic myelogenous leukemia and has more than 30% blasts in peripheral blood or bone marrow.

47. The method of claim 43, wherein the DNA methylation inhibitor is a cytidine analog.

48. The method of claim 47, wherein the cytidine analog is decitabine.

49. The method of claim 48, wherein decitabine is administered to the patient via an intravenous infusion per day at a dose ranging from 1 to 100 mg/m2.

50. The method of claim 49, wherein decitabine is administered to the patient via an intravenous infusion per day at a dose ranging from 2 to 50 mg/m2.

51. The method of claim 49, wherein decitabine is administered to the patient via an intravenous infusion per day at a dose ranging from 5 to 20 mg/m2.

52. A composition comprising:
a DNA methylation inhibitor; and
imatinib mesylate.

53. The composition of claim 52, wherein the DNA methylation inhibitor is a cytidine analog.

54. The composition of claim 52, wherein the DNA methylation inhibitor is cytosine arabinoside.

55. The composition of claim 52, wherein the DNA methylation inhibitor is decitabine.

56. The composition of claim 52, wherein the composition is formulated for intravenous or subcutaneous administration.

57. A treatment method comprising:
administering to a patient having chronic myelogenous leukemia and manifesting intolerance to imatinib mesylate, a therapeutically effective amount of a DNA methylation inhibitor which mitigates the imatinib mesylate intolerance.

58. The method of claim 57, wherein the patient has already manifested intolerance to imatinib mesylate within 6 months of the treatment with imatinib mesylate as defined by manifesting a symptom selected from the group consisting of hepatoxicity, fluid retention syndrome, neutropenia, hemorrhage, dyspepsia, dyspnea, diarrhea, muscle cramps, skin rash, fatigue, headache, nausea, vomiting, and thrombocytopenia.
